(19) United States
(12) Patent Application Publication
(10) Pub. No.: US 2009/0148870 A1
(43) Pub. Date: Jun. 11, 2009

(54) Rapid Detection of Mycobacterium Tuberculosis and Antimicrobial Drug Resistance

(76) Inventor: Daniel G. Ericson, Rochester, MN (US)

Correspondence Address: HUGH MCTAVISH;MCTAVISH PATENT FIRM, 429 BIRCHWOOD COURTS, BIRCHWOOD, MN, 55110, US

(21) Appl. No.: 12/227,466

(22) PCT Filed: May 18, 2007
(86) PCT No.: PCT/US07/11957

§ 371 Date: Nov. 18, 2008

Related U.S. Application Data
(60) Provisional application No. 60/801,365, filed on May 18, 2006.

(51) Int. Cl. G01N 33/569; C12M 1/00; C12Q 1/66

(52) U.S. Cl. 435/7.32; 435/287.2; 435/8

(57) ABSTRACT

The invention includes methods and kits for rapidly detecting tuberculosis or other mycobacterial infection in a sputum sample inexpensively and within minutes. It includes methods and kits for determining the species or phylogenetic group of mycobacterial infection. It includes methods and kits for determining the drug sensitivity of mycobacteria from a sputum sample inexpensively and within 1-3 days.

### BACKGROUND

In 1993, the World Health Organization (WHO) declared tuberculosis a global emergency. In 2002 there were an estimated 8.8 million new cases of tuberculosis worldwide, with an incidence rate of 141 per 100,000 population. The global incidence of tuberculosis is growing at approximately 1.1% per year, with much higher rates of growth in sub-Saharan Africa and in countries of the former Soviet Union. Developing countries suffer the brunt of the tuberculosis epidemic. Overall, it is estimated that 95% of the world's tuberculosis cases and 98% of the tuberculosis deaths occur in the developing world, and that tuberculosis causes more than 25% of avoidable deaths in the developing world (Maher, D. et al., 2002, A Strategic Framework to Decrease the Burden of TB/HIV, Geneva, Switzerland; World Health Organization).

Tuberculosis is caused by infection with the bacterium Mycobacterium tuberculosis. Globally, the emergence of multidrug resistant strains of M. tuberculosis is an increasing problem that adversely affects patient care and public health. It is estimated that one third of the global population, two billion people, are infected with M. tuberculosis. Current techniques for detection of M. tuberculosis infection, and susceptibility testing are time consuming and expensive. The hardest hit countries are typically the low income countries where testing the general population is not feasible due to cost constraints.

Effective treatment of tuberculosis requires the identification of Mycobacterium tuberculosis-infected patients and detection of drug resistance in all new patients. Biotechnology companies have developed rapid methods for tuberculosis diagnosis and detection of drug resistance; but all of the current methods have associated costs that would prohibit widespread use in the hardest hit developing countries.

To effectively stem the tuberculosis epidemic will require screening large numbers of people in the developing world for M. tuberculosis infection and treating those infected. Furthermore, to effectively treat those infected and to prevent the spread of multidrug resistant tuberculosis will require testing those infected to determine whether they are infected with a drug-resistant strain. For use in the developing world, the tests for tuberculosis infection and drug susceptibility should be inexpensive, simple, and fast. A test for M. tuberculosis infection in particular should preferably give a result within minutes, so that no follow-up with the patient is required to inform him that he is M. tuberculosis positive and to urge him to begin treatment.

Thus, new tests for M. tuberculosis infection are needed. New tests for determining whether those infected with M. tuberculosis are infected with drug-resistant or drug-sensitive strains are also needed. Preferably, the tests should be inexpensive enough to be used on a large scale in the developing world. Preferably, the tests will be rapid and reliable.

Other mycobacterial infections of the lungs, particularly M. avium complex, are also common in immunocompromised patients (Saubolle, M. A., 1989, Clin. Microbiol. News 11:113-117). M. avium complex is also a clinically significant pathogen in patients with chronic pulmonary disease (Kiehn, T. E. et al., 1987, J. Clin. Microbiol. 25:1551-1552). Accordingly, there is a need for rapid methods to detect mycobacterial infections generally, and tuberculosis and M. avium complex infections specifically, and to distinguish between them. M. avium complex is also commonly drug-resistant, and thus there is a need to test mycobacterial infections generally, and tuberculosis and M. avium complex infections specifically for drug sensitivity.

### SUMMARY

The invention provides a low-cost and very fast method of screening people for M. tuberculosis infection or infection with other mycobacteria. A person is asked to cough sputum into a receptacle. The sputum is passed over a small quantity of beads that bind any M. tuberculosis or other mycobacteria to their surface. The beads are then washed with a fluorescent or chemiluminescent stain that stains mycobacteria. Nonspecifically bound stain is then washed off, and the beads are placed in a hand-held light detector to detect any fluorescence (or chemiluminescence) from the beads. A high level of fluorescence indicates the presence of mycobacteria. The complete procedure can be carried out in less than 5 minutes. The reagents can be sufficiently cheap to allow a cost of potentially less than a dollar per test. Auramine O-rhodamine is a preferred stain for use in the procedure. It is an acid-fast fluorescent stain that is specific for mycobacteria. An inexpensive single-use disposable cartridge can be preloaded with the stain and any wash or counter-stain solutions used in the procedure to detect mycobacteria. The cartridge can be used to stain and wash cells bound to the beads.

Another embodiment of the invention provides a method of determining whether a mycobacteria-infected person is infected with drug-resistant or drug-sensitive mycobacteria (e.g., M. tuberculosis). In one embodiment, a sputum sample from a person is obtained and contacted with beads that bind mycobacteria. The beads are then divided into two or more groups and placed into mycobacterial growth medium. One group goes into a control vessel with medium containing no test agent. The other groups are placed into test vessels with medium containing one or more test agents. The vessels are then incubated for a period of time to allow mycobacterial growth in permissive conditions. Then the extent of growth is assayed by lysing the cells and contacting the lysate with luciferin and luciferase to produce luminescence that is proportional to the amount of ATP in the lysate. The amount of luminescence is compared between the control and test vessels to determine whether the test agents inhibited mycobacterial growth.

Thus, one embodiment of the invention provides a method of detecting mycobacterial or tuberculosis infection in a sputum sample of a patient comprising: (a) obtaining a sputum sample from a human; (b) contacting the sputum sample with a bacteria-binding apparatus having a surface that binds mycobacteria or a phylogenetic group of mycobacteria to isolate bound mycobacteria if any are present; (c) contacting the isolated mycobacteria with a fluorescent or chemiluminescent stain solution that stains mycobacteria or a phylogenetic group of mycobacteria; (d) washing the isolated mycobacteria with a wash solution to remove nonspecifically bound stain; and (e) detecting and quantifying light emission from the bound stain; wherein the bacteria-binding apparatus surface specifically binds mycobacteria or a phylogenetic group of mycobacteria, or the stain specifically stains mycobacteria or a phylogenetic group of mycobacteria; and wherein light emission above a threshold value is diagnostic of mycobacterial infection.

Another embodiment of the invention provides a method of determining whether a person afflicted with mycobacteria is infected with drug-resistant or drug-sensitive mycobacteria comprising:

(a) obtaining a sputum sample from the person;

(b) dividing the sputum sample or mycobacteria isolated from the sputum sample into 2 or more mycobacteria-containing fractions containing approximately equal numbers of mycobacteria;

(c) placing one of the mycobacteria-containing fractions into a control vessel containing a selective mycobacterial growth medium that allows growth of mycobacteria and inhibits growth of non-mycobacteria, wherein the medium lacks any test agent;

(d) placing each of the one or more other mycobacteria-containing fractions into a test vessel containing the selective medium supplemented with one or more test anti-mycobacteria agents;

(e) incubating the control and test vessels for a period of time sufficient to allow mycobacterial growth in at least the control vessel; and

(f) assaying for mycobacterial cell numbers in the control and test vessels by a process comprising:
(i) lysing mycobacteria from the vessels to release mycobacterial ATP, and contacting mycobacterial ATP with luciferin and luciferase in an assay solution for each vessel; and
(ii) detecting and quantifying light emitted from the assay solution for each vessel, wherein the quantity of light emitted is proportional to the number of mycobacterial cells in the vessel after the growth period.

Another embodiment of the invention provides a combined method of detecting mycobacterial infection in a sputum sample and determining drug sensitivity of the mycobacterial infection comprising:

(a) obtaining at least one sputum sample from a human;

(b) contacting one of the at least one sputum samples with a bacteria-binding apparatus having a surface that binds mycobacteria or a phylogenetic group of mycobacteria to isolate bound mycobacteria if any are present;

(c) contacting the isolated mycobacteria with a fluorescent or chemiluminescent stain solution that stains mycobacteria or a phylogenetic group of mycobacteria;

(d) washing the isolated mycobacteria with a wash solution to remove nonspecifically bound stain, and

(e) detecting and quantifying light emission from the bound stain;

wherein the bacteria-binding apparatus surface specifically binds mycobacteria or a phylogenetic group of mycobacteria, or the stain specifically stains mycobacteria or a phylogenetic group of mycobacteria; and wherein light emission above a threshold value is diagnostic of mycobacterial infection;

(f) when the sputum sample is infected with mycobacteria, dividing one of the at least one sputum samples infected with mycobacteria, or mycobacteria isolated from the sputum sample, into two or more mycobacteria-containing fractions containing approximately equal numbers of mycobacteria;

(g) placing one of the mycobacteria-containing fractions into a control vessel containing a selective mycobacterial growth medium that allows growth of mycobacteria and inhibits growth of non-mycobacteria, wherein the medium lacks any test agent;

(h) placing each of the one or more other mycobacteria-containing fractions into a test vessel containing the selective medium supplemented with one or more test anti-mycobacteria agents;

(i) incubating the control and test vessels for a period of time sufficient to allow mycobacterial growth in at least the control vessel; and

(j) assaying for mycobacterial cell numbers in the control and test vessels by a process comprising:
(1) lysing mycobacteria from the vessels to release mycobacterial ATP, and contacting mycobacterial ATP with luciferin and luciferase in an assay solution for each vessel; and
(2) detecting and quantifying light emitted from the assay solution for each vessel, wherein the quantity of light emitted is proportional to the number of mycobacterial cells in the vessel after the growth period.

Another embodiment of the invention provides a combined method of detecting mycobacterial infection in a sputum sample and determining the presence or absence of a phylogenetic group of mycobacteria in the infection comprising: (a) obtaining at least one sputum sample from a human; (b) contacting one sputum sample of the at least one sputum samples with a bacteria-binding apparatus having a surface that binds mycobacteria to isolate bound mycobacteria if any are present; (c) contacting the isolated mycobacteria with a fluorescent or chemiluminescent stain solution that stains mycobacteria; (d) washing the isolated mycobacteria with a wash solution to remove nonspecifically bound stain; and (e) detecting and quantifying light emission from the bound stain; wherein the bacteria-binding apparatus surface specifically binds mycobacteria or the stain specifically stains mycobacteria; and wherein light emission above a threshold value is diagnostic of mycobacterial infection; (f) obtaining a sample of mycobacteria, with or without growth amplification, from one of the at least one sputum samples of the human wherein the sputum sample is infected with mycobacteria; (g) contacting the mycobacterial sample with a dye-conjugated antibody that specifically recognizes a phylogenetic group of mycobacteria; and (h) detecting recognition or lack of recognition of the phylogenetic group of mycobacteria in the mycobacterial sample by the dye-conjugated antibody.

Another embodiment of the invention provides a sputum receptacle containing: a vessel for receiving sputum; and a bacteria-binding apparatus (e.g., a collection of beads) functionally coupled to the vessel and having a surface that binds mycobacteria or a phylogenetic group of mycobacteria (e.g. Mycobacterium tuberculosis).

Another embodiment of the invention provides a handheld disposable cartridge for use in detecting mycobacteria that includes: (i) a receptacle holding (ii) a bacteria-binding apparatus having a surface that binds mycobacteria; and (iii) a fluorescent or chemiluminescent stain that stains mycobacteria functionally coupled to the receptacle to stain mycobacteria held by the apparatus in the receptacle, wherein the bacteria-binding apparatus surface specifically binds mycobacteria or a phylogenetic group of mycobacteria, or the stain specifically stains mycobacteria or a phylogenetic group of mycobacteria.

Another embodiment of the invention provides a kit for detecting mycobacteria. The kit includes two components. The first component is a disposable sputum receptacle comprising: (i) a vessel for receiving sputum; and (ii) a bacteria-binding apparatus functionally coupled to the vessel and having a surface that binds mycobacteria. The second component is a handheld disposable cartridge for use in detecting Mycobacterium tuberculosis comprising: (i) a receptacle for holding the bacteria-binding apparatus having a surface that binds mycobacteria; and (ii) a fluorescent or chemiluminescent stain that stains mycobacteria functionally coupled to the receptacle to stain mycobacteria held by the apparatus in the receptacle. In the kit, the stain specifically stains mycobacteria or a phylogenetic group of mycobacteria (e.g., M. tuberculosis) or the surface within the vessel specifically binds mycobacteria or a phylogenetic group of mycobacteria.

Another embodiment of the invention provides a kit for use in determining whether a person infected with mycobacteria is infected with drug-resistant or drug-sensitive mycobacteria. The kit includes a vessel for receiving sputum from a person; particles having a surface that binds mycobacteria; and luciferase and luciferin.

Another embodiment of the invention provides a kit for detection of a phylogenetic group of mycobacteria comprising: (a) a dye-conjugated antibody that specifically recognizes a phylogenetic group of mycobacteria; and (b) a substrate comprising (i) an immobilized antibody that binds to mycobacteria bound to the antigen-binding site of the dye-conjugated antibody or (ii) an immobilized antigen recognized by the dye-conjugated antibody.

### BRIEF DESCRIPTION OF THE DRAWINGS

FIG. 1 provides a view of a sputum receptacle of the invention.

FIG. 2 shows an example of a cartridge for use in detecting mycobacteria from a sputum sample.

FIG. 3 shows another example of a cartridge for use in detecting mycobacteria.

FIG. 4 is a drawing of a light detector for use in detecting mycobacteria from a sputum sample by the methods of the invention.

FIG. 5 is a drawing of a lateral flow immunoassay device for determining the presence or absence of a particular species or other phylogenetic group of mycobacteria in a sample.

### DETAILED DESCRIPTION

### Definitions

The term “wash” as used herein includes contacting cells or other material with a destain solution to remove nonspecifically bound stain. For instance, when using an acid-fast stain to stain the cell wall of mycobacteria, the term “wash” includes contacting the cells or other material with an acid-alcohol solution to remove nonspecifically bound stain.

The term “phylogenetic group” of mycobacteria refers to a single species, such as M. tuberculosis, a strain, or a group of species such as M. avium complex, which is generally divided into two species M. avium and M. intracellulare, that is less than the whole genus of mycobacteria.

The term “luminescence” includes fluorescence, chemiluminescence, and other forms of light emission from an energy source.

### DESCRIPTION

One embodiment of the invention provides a method of detecting mycobacterial or tuberculosis infection in a sputum sample of a patient comprising: (a) obtaining a sputum sample from a human; (b) contacting the sputum sample with a bacteria-binding apparatus having a surface that binds mycobacteria or a phylogenetic group of mycobacteria (e.g. M. tuberculosis or M. avium complex) to isolate bound mycobacteria if any are present; (c) contacting the isolated mycobacteria with a fluorescent stain solution that stains mycobacteria or a phylogenetic group of mycobacteria; (d) washing the isolated mycobacteria with a wash solution to remove nonspecifically bound stain; and (e) detecting and quantifying fluorescence from the bound stain; wherein the bacteria-binding apparatus surface specifically binds mycobacteria or a phylogenetic group of mycobacteria, or the fluorescent stain specifically stains mycobacteria or a phylogenetic group of mycobacteria; and wherein light emission above a threshold value is diagnostic of bacterial infection.

In a preferred embodiment, the bacteria-binding apparatus having a surface that binds mycobacteria or a phylogenetic group of mycobacteria comprises or is a collection of beads having a surface that binds mycobacteria or a phylogenetic group of mycobacteria (e.g. M. tuberculosis or M. avium complex). It can be, for instance, a chamber holding a collection of beads. The chamber wall may be permeable to fluid and to bacteria. The bacteria-binding surface could also be the surface of a vessel for receiving sputum. It could be the surface of gold particles, e.g., gold particles coated with a monoclonal antibody that recognizes M. tuberculosis.

In a preferred embodiment, the isolated mycobacteria are contacted with the stain solution and washed with the wash solution while bound to the bacteria-binding apparatus surface, and the fluorescence is detected and quantified from the bacteria-binding apparatus surface. For instance, all these steps may be done while the bacteria are bound to bacteria-binding beads.

It is also possible to elute the M. tuberculosis from the binding surface used to isolate them before any or all of the steps of staining with a fluorescent stain, washing, and detecting fluorescence.

One of the advantages of the method is that it can be performed in a short time. In specific embodiments, steps (c) through (e) are performed in less than 10, less than 5, less than 3, or less than 2 minutes.

In specific embodiments, steps (b) through (e) are performed in less than 10, less than 5, less than 3, or less than 2 minutes.

In one embodiment of the method of detecting mycobacterial infection, after the steps of contacting the mycobacteria with a stain solution and washing the isolated mycobacteria with a wash solution, the method includes contacting the isolated mycobacteria with a counter-stain solution.

It is not necessary to culture mycobacteria from the sputum sample in order to amplify them before taking the steps to detect mycobacteria from the sputum sample.

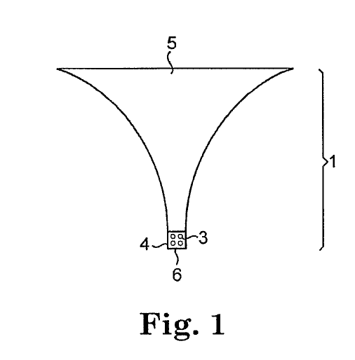

In one embodiment with bacteria-binding beads, the beads are part of a sputum receptacle. In FIG. 1 the sputum receptacle 1 includes a vessel for receiving sputum 2 and beads 3 having a surface that binds mycobacteria or a phylogenetic group of mycobacteria. The beads 3 are held in a detachable bead chamber 4.

A person is asked to expectorate sputum into the vessel 2. The sputum is washed down to contact the beads 3 in the bead chamber 4.

Sputum is thick and viscous and comes from the lungs. It is typically brought from the lungs by coughing. It must be distinguished from saliva, which is considerably thinner. The tests herein require sputum. To provide sputum samples, subjects should breathe two deep breaths and then cough until they are able to expectorate a thick viscous sputum sample. Steps can be taken to assist a person in providing a sputum sample. Breathing hot moist air will thin the mucus of the airway passages and make it easier to cough up a sputum sample. A subject may breathe mist from a 3-15% salt solution for 5-15 minutes with forceful coughing to provide a sputum sample. The mist can be provided by a jet hand-held nebulizer or an ultrasonic nebulizer.

In the embodiment shown in FIG. 1, the vessel 2 is a funnel that tapers from a wider-dimensioned outer opening 5 to a narrower-dimensioned inner opening 6. The bottom of the funnel and the bead chamber may allow fluid passage, so the sputum is washed over the beads 3 to contact them. Water or saline can be used to wash the sputum. A solution such as SPUTASOL that solubilizes sputum may also be used. SPUTASOL includes dithiothreitol in a saline solution.

In one embodiment, the bead chamber 4 is detachable from the receptacle 1.

In a specific embodiment, the bead chamber 4 may be attached adjacent to the inner opening 6 of the funnel, as shown in FIG. 1.

A bead chamber may also be placed loose in the vessel for receiving sputum, where the bead chamber is permeable to mycobacteria to allow mycobacteria to contact beads in the chamber. In this case also the bead chamber is a bacteria-binding apparatus that is functionally coupled to the vessel for receiving sputum.

Beads may also be loose in a vessel for receiving sputum, rather than trapped in a chamber.

Thus, the bacteria-binding apparatus may comprise a collection of beads having a surface that binds mycobacteria or a phylogenetic group of mycobacteria.

In a preferred embodiment, the fluorescent or chemiluminescent stain is an acid-fast fluorescent or chemiluminescent stain. Auramine-O-rhodamine is a preferred acid-fast fluorescent stain. The acid-fast stains specifically stain mycobacteria.

In a particular embodiment, the bead chamber is adapted to engage with a disposable cartridge for use in detecting mycobacteria or a phylogenetic group of mycobacteria. The cartridge includes a receptacle for receiving the bead chamber, and in a specific embodiment includes an acid-fast fluorescent stain.

In a particular embodiment, the sputum receptacle is disposable, i.e., adapted for single-use.

The bacteria-binding apparatus having a surface that binds mycobacteria can have a surface that non-specifically binds all bacteria, such as GENPOINT beads (Oslo, Norway). Alternatively, the surface may selectively bind mycobacteria or more specifically M. tuberculosis or another phylogenetic group of mycobacteria. For instance, gold particles can be coated with antibodies specific for M. tuberculosis (BioAssay Works, LLC, Ijamsville, Md., bioassayworks.com).

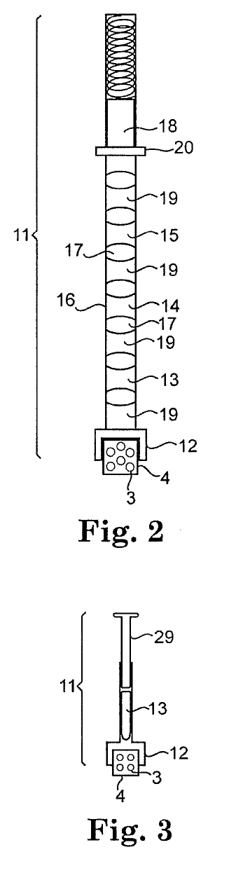

Another embodiment of the invention provides a handheld disposable cartridge for use in detecting Mycobacterium tuberculosis that includes: a receptacle for holding a bacteria-binding apparatus having a surface that binds mycobacteria; and a fluorescent stain that stains mycobacteria functionally coupled to the receptacle to stain mycobacteria held by the apparatus in the receptacle. An embodiment of this cartridge is shown in FIG. 2. The cartridge show in FIG. 2 is handheld disposable pen 11. It includes a receptacle 12 for holding a detachable bead chamber 4 containing beads 3 with a surface that binds mycobacteria. In the barrel 16 of the pen the cartridge contains a series of solutions for staining and destaining mycobacterial cells on the beads with a fluorescent or chemiluminescent stain. The solutions are arranged in stacked zones. The stacked zones can be separated by air bubbles 17 or barriers that are perforated by pressure. The solutions in stacked zones in this example in order are water 19, auramine O-rhodamine 13, water 19, acid-alcohol 14, water 19, erichrome black T 15, and water 19. Auramine O-rhodamine is an acid-fast fluorescent stain that stains mycobacteria specifically. Acid-alcohol removes the stain from non-acid-fast bacterial cell walls and other debris. Erichrome black T is a counterstain that reduces the background signal.

In particular embodiments of the cartridge, the stain specifically stains mycobacteria, M. tuberculosis, or M. avium complex.

The cartridge in FIG. 2 also includes a spring-loaded plunger 18, shown with a release mechanism 20. The spring-loaded plunger can be mechanically activated. In one embodiment, the plunger is designed so that upon release, it drives the solutions over the beads over about 60 seconds, or another predetermined period of time.

Another embodiment of the cartridge is shown in FIG. 3. In FIG. 3, the cartridge is a syringe 11 having a receptacle 12 for holding a bead chamber 4, and holding a solution of a fluorescent stain such as auramine O-rhodamine 13. To execute the same staining procedure—contacting the beads in sequence with water, auramine O-rhodamine solution, water, acid-alcohol, water, erichrome black T solution, and water—the same syringe can be loaded with the appropriate solution and used to discharge the solution over the beads, before loading it with the next solution. Alternatively, separate syringes could be used, where one contains water, one an auramine-O-rhodamine solution, one an acid-alcohol solution, and one an erichrome black T solution, where each syringe includes a receptacle for receiving the beads. The beads would be first washed with the water solution, then the auramine-O-rhodamine solution, then water, then acid-alcohol, then erichrome black T, and then water. The syringe 11 shown in FIG. 3 has a plunger 29.

Other fluorescent or chemiluminescent stains could also be used—for instance, a fluorescent stain coupled to an antibody that specifically binds to mycobacteria or more specifically to M. tuberculosis.

If the beads bind all bacteria, then the stain should specifically stain mycobacteria or M. tuberculosis. If the beads specifically bind only mycobacteria or M. tuberculosis, then a non-specific fluorescent or chemiluminescent stain that would stain other types of bacteria can be used.

A class of stains that specifically stain mycobacteria is the acid-fast stains. These are specific for the unusual cell wall structure of mycobacteria. An example of a fluorescent acid-fast fluorescent stain is auramine-O-rhodamine. Another acid-fast stain that is not fluorescent or chemiluminescent is carbol-fuchsin, a mixture of basic fuchsin and phenol. A chemiluminescent acid-fast stain can by synthesized by coupling a chemiluminescent agent such as luminol with basic fuchsin. The fluorescent or chemiluminescent stain can also be a fluorescent or chemiluminescent group conjugated to an antibody that specifically recognizes mycobacteria or a phylogenetic group of mycobacteria.

In specific embodiments, as in FIG. 2, the fluorescent or chemiluminescent stain is in a solution and the cartridge further comprises a wash solution functionally coupled to the receptacle to wash stained mycobacteria held by the apparatus in the receptacle.

In specific embodiments, as shown in FIG. 2, the cartridge 11 comprises a fluorescent or chemiluminescent stain solution 13, a wash solution 14 and 19, and a counter-stain solution 15. The counter-stain solution is functionally coupled to the receptacle to count-stain material held by the apparatus in the receptacle (e.g., bacteria that are not mycobacteria).

In specific embodiments, as shown in FIG. 2, the fluorescent stain solution and the wash solution are in separate stacked zones in the cartridge.

In specific embodiments, the fluorescent stain solution and the wash solution are held in discrete compartments in the cartridge.

In a specific embodiment shown in FIG. 2, the cartridge includes a spring-loaded pump mechanism 18 functionally linked to the stacked zones to pump the stacked zone solutions over the beads or other bacteria binding apparatus in the cartridge when the beads are held in the receptacle 12.

The common mycobacterial infections of humans are leprosy, caused by M. leprae, tuberculosis, caused by M. tuberculosis, and M. avium complex infections. The lungs are not the natural site of M. leprae infection, and M. avium infection is considerably less common than M. tuberculosis infection in many less developed parts of the world. So a sputum sample positive for mycobacterial infection in some circumstances can be taken as strongly indicative of tuberculosis.

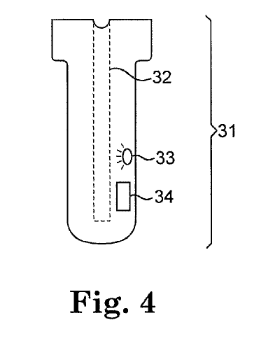

After staining and destaining any mycobacterial cells on the surface of the bacteria-binding apparatus, the bacteria-binding apparatus (in one embodiment a collection of beads) is transferred to a light detector. The light detector can be handheld, and can be a fluorescence detector or a chemiluminescence detector. An example of a fluorescence detector 31 is shown in FIG. 4. It includes an excitation lamp 33 capable of exciting the fluorescent stain, and a light-detector system 34 capable of detecting and quantifying fluorescence from the fluorescent stain. The detector also has a receptacle 32 adapted to hold the bacteria-binding apparatus used to bind mycobacteria from the sputum. The receptacle 32 can, for instance, be designed to hold the cartridge 11 shown in FIG. 2 with the bacteria-binding beads held on the cartridge. To detect chemiluminescence, the excitation lamp is not necessary.

One embodiment of the invention is a kit for detecting mycobacteria (e.g. M. tuberculosis). The kit includes two components. The first component is a disposable sputum receptacle comprising: (i) a vessel for receiving sputum; and (ii) a bacteria binding apparatus (e.g., a collection of beads) functionally coupled to the receptacle and having a surface that binds mycobacteria, as shown in FIG. 1, for instance. The second component is a handheld disposable cartridge for use in detecting mycobacteria comprising: (i) a receptacle for holding beads having a surface that binds mycobacteria; and (ii) a fluorescent or chemiluminescent stain that stains mycobacteria, as shown in FIG. 2, for instance. In the kit, the fluorescent stain specifically stains mycobacteria or a phylogenetic group of mycobacteria (e.g., M. tuberculosis) or the bacteria-binding apparatus surface specifically binds mycobacteria or a phylogenetic group of mycobacteria.

In a particular embodiment, the kit further includes a light-detector system capable of detecting and quantifying light emission from the fluorescent or chemiluminescent stain. The light detector system may be a fluorescence detector having an excitation lamp capable of exciting the fluorescent stain. Quantification of light emission from the fluorescent or chemiluminescent stain could involve determining a numerical value for the intensity of the light emission, or determination of whether the intensity of light emission exceeds a threshold value.

The first aspect of the present invention involves detecting mycobacterial infection, e.g., M. tuberculosis infection. A second aspect involves determining whether a person afflicted with mycobacterial infection or specifically tuberculosis is infected with drug-resistant or drug-sensitive mycobacteria or M. tuberculosis.

The method of determining drug sensitivity involves first obtaining a sputum sample from the person infected with mycobacteria, and then dividing the sputum sample or mycobacteria isolated from the sputum sample into two or more bacteria-containing fractions containing approximately equal numbers of mycobacteria. A preferred method of dividing the mycobacteria into fractions is to contact the sputum sample with beads having a surface that binds mycobacteria, and then dividing the beads into two or more groups of approximately equal total surface area of the beads, wherein the beads contain approximately equal numbers of bound mycobacteria.

After the sputum or mycobacteria isolated from the sputum are divided into fractions, one of the fractions is placed into a control vessel containing a selective mycobacterial growth medium that allows growth of mycobacteria and inhibits growth of non-mycobacteria, where the control vessel medium lacks any test anti-mycobacteria or anti-tuberculosis agent. So it permits mycobacterial growth. Each of the one or more other fractions is placed into its own test vessel. Each of the test vessels contains the same medium, supplemented with a test anti-mycobacteria agent or a combination of agents.

In a preferred embodiment, particularly when testing for M. tuberculosis drug resistance, one test vessel contains isoniazid, and another contains rifampicin. Those are the two most important anti-tuberculosis drugs. In another embodiment, one test vessel contains both isoniazid and rifampicin.

In a preferred embodiment, mycobacteria from sputum is bound to beads, the beads are divided into two or more fractions, and the beads of one fraction are placed in the control vessel, and the beads of each of the other fractions are placed into their own test vessel. Gen-Point C version BUGS-N-BEADS™ beads have been used. Preferably the mycobacteria are not eluted from the beads but just placed in the growth medium on the beads. It has been determined that mycobacteria from new growth are in the liquid medium, but the originally isolated bacteria from the sputum sample remain bound to the beads. Thus, even allowing only time for a single doubling can result in a large difference in the amount of mycobacteria found in the liquid medium in permissive medium versus the amount found in the liquid medium with rifampin or isoniazid if the isolate is sensitive to rifampin and isoniazid. We have had success detecting resistance or sensitivity to rifampin and isoniazid with culture periods of 72 hours or less, and in some cases culture for 24 hours.

After placing the bacteria-containing fractions in the control vessel and the test vessels, the vessels are incubated for a period of time sufficient to allow mycobacterial (e.g., M. tuberculosis) growth in the control vessel. In particular embodiments, this period of time is 3 days or less, 2 days or less, or 24 hours or less.

Then mycobacterial cells numbers are assayed in the control and test vessels by a process involving contacting mycobacteria from the vessels with a cell lysing agent and luciferin/luciferase to lyse the mycobacteria and then to contact ATP released from the lysed mycobacteria with luciferin and luciferase in an assay solution for each vessel.

The mycobacteria need not be isolated from the growth medium prior to assaying for mycobacterial cell numbers. They can be assayed directly in the growth medium. This saves cost and reduces handling steps. Thus, preferably the lysing agent, luciferin, and luciferase are added directly to the liquid medium, and the liquid medium containing these agents is placed in a light detector to detect light emission. Where mycobacteria are added to the culture vessels bound to a binding surface, such as the binding bead binding surface, in some embodiments the liquid medium is withdrawn from the binding surface prior to adding the lysing agent, luciferin, and luciferase to assay for cell numbers. This may increase the difference in growth between permissive and restrictive growth vessels, because the originally bound cells may remain bound to the substrate surface, while new growth is in the liquid medium. However, if the test agent kills the originally bound cells, rather than merely preventing their growth, and the cells have no ATP, then separation of the liquid medium does not provide an advantage.

In one embodiment, the cells are lysed by contacting the mycobacteria with a single solution containing a lysing agent, luciferin, and luciferase.

Luciferase catalyzes a light emitting reaction with ATP and luciferin as substrates. The reaction is shown below, where E=luciferase and LH₂=luciferin.

E+LH₂+ATP+Mg⁺⁺--->E-LH₂-AMP+Mg⁺⁺+PPᵢ

E-LH₂-AMP+O₂--->E+CO₂+AMP+oxyluciferin+photon

Provided there is excess luciferase and luciferin, and oxygen is available, the reaction is proportional to ATP concentration. ATP concentration, in turn, is proportional to the number of viable bacterial cells that are lysed. Thus, light emission in this assay is proportional to the number of viable mycobacterial cells.

Cell number can also be assayed by staining the mycobacteria with a fluorescent or chemiluminescent stain, but this would require binding the mycobacterial cells to a surface so that unbound stain could be washed off. Lysing the cells and assaying for ATP with luciferase and luciferin can be done directly in liquid medium without a binding and washing step. This reduces reagent use, handling steps, and time.

A selective medium is used for the growth of the mycobacteria in the assay. An example is the Middlebrook selective medium 7H11, containing the following ingredients per liter.

Magnesium sulfate 0.05 g.
Ferric ammonium citrate 0.04 g.
Sodium citrate 0.4 g.
Ammonium sulfate 0.5 g.
Monosodium glutamate 0.5 g.
Disodium phosphate 1.5 g.
Monopotassium phosphate 1.5 g.

### Pyridoxine 1.0 mg.

Zinc sulfate 1.0 mg.
Copper sulfate 1.0 mg.

### Biotin 0.5 mg.

Calcium chloride 0.5 mg.
Malachite green 0.25 mg.
OADC enrichment 100 ml.

### Glycerol 5 ml.

Pancreatic digest of casein 1.0 g.
Polymyxin B 200,000 units.

### Carbenicillin 50 mg.

### Amphotericin B 10 mg.

Trimethoprim lactate 20 mg.

OADC enrichment contains per liter, 8.5 g sodium chloride, 50 g bovine albumin, 20 g dextrose, 30 mg catalase, and 0.6 ml oleic acid.

The polymyxin B, carbenicillin, amphotericin B, and trimethoprim lactate are antibiotics and antifungals that inhibit the growth of non-mycobacteria.

Mycobacterial cells can be lysed by contact with lysing agents such as detergents, acids such as trichloroacetic acid, or bases. Trichloroacetic acid and perchloric acid, and probably other acids, have the advantage of denaturing bacterial apyrase, which otherwise can hydrolyze the released ATP (9, 10). Bacterial cells can also be lysed by sonication, heating, contact with particles (e.g., glass beads), freeze-thaw, organic solvents (e.g., chloroform, phenol, or n-butanol), enzymes (e.g., lysozyme), or french press. Combinations of two or more of the above lysing methods or agents may also be used.

If acid or base is used to lyse the mycobacterial cells, the pH may need to be adjusted after the lysis step before adding, or simultaneously with adding, luciferase and luciferin.

The mycobacteria can also be lysed with a lysing agent in a mixture with luciferase and luciferin if the lysing agent is compatible with luciferase and luciferin. For instance, BACTITER-GLO reagent from Promega (Madison, Wis.) contains detergents to lyse bacteria in a mixture with luciferase and luciferin.

Luminescence from the assay with lysed mycobacteria can be detected with a light detector. Light detector systems for detecting bioluminescence as well as chemiluminescence are described in U.S. patent application Ser. No. 10/968,203.

Another embodiment of the invention provides a kit for use in determining whether a person infected with mycobaceteria (e.g., M. tuberculosis) is infected with drug-resistant or drug-sensitive mycobacteria The kit includes a vessel for receiving sputum from a person; a bacteria-binding apparatus (e.g., a collection of particles or beads) having a surface that binds mycobacteria; and luciferase and luciferin.

In particular embodiments, the kit also includes a mycobacterial selective growth medium.

In another particular embodiment, the kit includes one or more test anti-mycobacterial (e.g., anti-tuberculosis) agents.

In another embodiment, the kit includes a mycobacterial lysing agent.

In another embodiment, the kit includes two or more containers for holding growth medium and culturing mycobacteria.

In another embodiment, the kit includes a light detector system (e.g., a device or system capable of detecting and quantifying fluorescence or chemiluminescence or both).

The methods of the invention can also involve diagnosing the type of mycobacterial infection in a sputum sample by determining the presence or absence of a phylogenetic group of mycobacteria in a sputum sample infected with mycobacteria. Infection with mycobacteria of an unknown species can be determined by using a binding surface that binds all mycobacteria and a stain that stains all mycobacteria, such as an acid-fast fluorescent or chemiluminescent stain. Which species or other phylogenetic group of mycobacteria is involved in the infection can then be determined by assaying for binding to an antibody that specifically recognizes the phylogenetic group of mycobacteria. The antibody can be monoclonal or polyclonal or a collection of monoclonal or polyclonal antibodies. If the antibody is immobilized to a bacteria-binding apparatus surface, e.g., a bead surface, a sample containing mycobacteria can be contacted with the surface to isolate mycobacteria of the phylogenetic group recognized by the antibody. The bound mycobacteria can be stained with a fluorescent or chemiluminescent stain. Nonspecifically bound stain is washed off. And light is detected and quantified to determine the presence or absence of the phylogenetic group of mycobacteria. The sample containing mycobacteria can be a sputum sample or a sample of mycobacteria amplified or isolated from a sputum sample.

In a preferred embodiment to detect a mycobacterial phylogenetic group, an antibody to the phylogenetic group is conjugated to a dye. The dye-conjugated antibody is contacted with the mycobacterial sample, and recognition or lack of recognition of the phylogenetic group of mycobacteria in the sample by the antibody is detected.

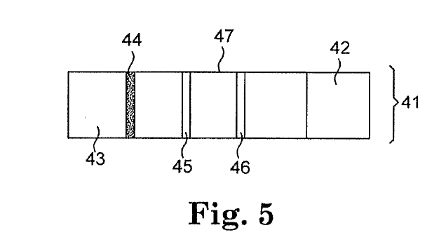

A lateral flow immunoassay is one method of carrying this out. Lateral flow immunoassays are perhaps most commonly used to test for pregnancy by detecting pregnancy hormones with antibodies. One embodiment is shown in FIG. 5. Lateral flow immunoassay device 41 is shown. To detect the mycobacterial phylogenetic group, a dye-conjugated antibody is mixed with the sample. The dye-conjugated antibody may be immobilized on gold or latex particles. This amplifies the signal since multiple antibody and dye molecules are on the same particle. In the embodiment of FIG. 5, the device 41 includes a substrate 47 that can be, for instance, nitrocellulose. The dye-conjugated antibody is impregnated at area 44 of a substrate 47. A fluid sample is placed at area 43 of the substrate 47. The sample can be placed directly in contact with the dye-conjugated antibody or may migrate to contact the antibody. The sample contacts the dye-conjugated antibody in area 44, and then by lateral fluid flow, the sample and the dye-conjugated antibody migrate toward an absorbent pad 42 at the other end of the device 41. Two strips of immobilized antibody are located at positions 45 and 46. The first strip 45 contains immobilized antibody that recognizes the same antigen as the dye-conjugated antibody. Thus, if the dye-conjugated antibody has bound antigen, this immobilized antibody also binds to the bound antigen, and the dye-conjugated antibody is bound to the first strip 45 as a dye-conjugated antibody-antigen-immobilized antibody sandwich. A second strip 46 contains immobilized anti-Fc-antibody that recognizes the Fc portion of the dye-conjugated antibody. If the sample contains no antigen recognized by the dye-conjugated antibody, the dye-conjugated antibody has no bound antigen and thus does not bind as a sandwich to the immobilized antibody of the first strip 45 and it passes by the first strip 45. It then reaches the second strip 46 where it is bound by the anti-Fc antibody. Thus, when the sample contains antigen recognized by the dye-conjugated antibody, in this case mycobacteria of a particular phylogenetic group, the dye-conjugated antibody accumulates on the first strip 45. If the sample does not contain the antigen, the dye-conjugated antibody accumulates on the second strip 46. The dye can be detected visually as a colored band. Or if the dye is fluorescent or chemiluminescent, it can be detected by light emission.

Other variations of the lateral flow immunoassay are also possible. In the above-described lateral flow immunoassay, an immobilized antibody that binds to mycobacteria bound to the antigen-binding site of the dye-conjugated antibody is used to detect dye-conjugated antibody recognition of the mycobacteria. In a variation, the antigen recognized by the dye-conjugated antibody is immobilized to a strip. If the dye-conjugated antibody has already bound to antigen in the sample, it has no antigen binding sites available to bind to the immobilized antigen and it does not bind to the immobilized antigen. But if the sample does not contain the phylogenetic group of mycobacteria being tested for, the dye-conjugated antibody has no antigen bound, and it is able to bind to the immobilized antigen and accumulate at a strip of the immobilized antigen.

Bioassayworks, LLC, (Ijamsville, Md.; www.bioassayworks.com) prepares gold-antibody conjugates and lateral flow immunoassay kits.

Thus, one embodiment of the invention provides a kit for detection of a phylogenetic group of mycobacteria containing: (a) a dye-conjugated antibody that specifically recognizes a phylogenetic group of mycobacteria; and (b) a substrate comprising (i) an immobilized antibody that binds to mycobacteria bound to the antigen-binding site of the dye-conjugated antibody or (ii) an immobilized antigen recognized by the dye-conjugated antibody.

### EXAMPLES

### Example 1

### Detecting Mycobacteria in a Sputum Sample

A sputum sample is obtained from a human subject by asking the subject to cough up a sputum sample and expectorate it into a small disposable paper cup. If necessary, the subject may be asked to breathe mist from a 15% hypertonic saline solution produced by a hand-held nebulizer for 5-15 minutes.

A 1 ml 2 mM dithiothreitol, 100 mM NaCl solution in water is added to the cup and mixed with the sputum sample to thin it. Gen-Point C version BUGS-N-BEADS™ beads (50 μl) are added and mixed with the sample with agitation for 30 seconds to allow bacteria in the sample to bind to the beads. The beads bind mycobacteria and many other types of bacteria.

The beads are washed with water in the cup and the wash is discarded.

Next the beads are incubated in 100 μl of heated auramine-O-rhodamine solution at 60° C. for 5 minutes. Auramine-O-rhodamine solution contains 1.5 g auramine O, 0.75 rhodamine B, 10 ml melted phenol, and 90 ml water.

The auramine-O-rhodamine solution is removed and the beads are washed with water. The beads are then washed with 1% sulfuric acid solution in water. The beads are then washed a further two times in water. The beads are then withdrawn and placed in a fluorescence detector. One fluorescence detector that may be used is the Turner Biosystems PICOFLUOR hand-held detector (Sunnyvale, Calif.). The absorbance peak of rhodamine B is 543 nm and auramine O's absorbance peak is 434 nm.

Fluorescence is quantified. For comparison and as a control, the same procedure is followed without a sputum sample, and the control fluorescence is quantified.

### Example 2

### Measuring Drug Resistance of Mycobacteria from a Sputum Sample

From a person testing positive for mycobacteria by the procedure of Example 1, another sputum sample is obtained as described in Example 1. The sputum sample is thinned by mixing with 1 ml of 2 mM DTT, 100 mM NaCl. The thinned sample is incubated for 30 seconds with 50 μl Gen-Point C version BUGS-N-BEADS™ beads to bind mycobacteria. (The beads will also bind other types of bacteria present in the sample.) The beads are then divided into three equal aliquots.

One aliquot of beads is placed in a culture tube containing 0.8 ml of Middlebrook selective medium 7H11. Another aliquot is placed in a culture tube containing 0.8 ml of Middlebrook selective medium 7H11 supplemented with 0.2 μg/ml isoniazid (0.2-2.0 μg/ml may be used, depending on the degree of resistance to be tested for). A third equal aliquot is placed in a culture tuber containing 0.8 ml of Middlebrook selective medium 7H11 supplemented with 1.0 μg/ml rifampin.

The tubes are incubated at 37° C. for 1-3 days in a 5-10% CO₂ atmosphere. At the end of the incubation period, the liquid medium from each tube is separately collected.

One-hundred microliters of 0.1% trichloroacetic acid in water heated to 60° C. is added to each of the separate growth medium samples to lyse mycobacteria.

A luciferin-luciferase solution (200 microliters, containing 0.2 μg luciferase, 2 μg luciferin, in 50 mM sodium phosphate pH 7.5) is added to the lysate mixture, and the mixture is immediately placed in a luminometer. Luminescence is quantified. A custom-made luminometer with a photomultiplier tube as described in U.S. patent application Ser. No. 10/968,203 may be used. A suitable commercially available luminometer is Zylux FB12.

The luminescence level is compared between the control vessel, the isoniazid vessel, and the rifampin vessel to determine whether the mycobacteria are resistant to either isoniazid or rifampin.

All patents, patent documents, and other references cited are incorporated by reference.

What is claimed is:

1. A method of detecting mycobacterial infection in a sputum sample of a patient comprising:
(a) obtaining a sputum sample from a human;
(b) contacting the sputum sample with a bacteria-binding apparatus having a surface that binds mycobacteria or a phylogenetic group of mycobacteria to isolate bound mycobacteria if any are present;
(c) contacting the isolated mycobacteria with a fluorescent or chemiluminescent stain solution that stains mycobacteria or a phylogenetic group of mycobacteria;
(d) washing the isolated mycobacteria with a wash solution to remove nonspecifically bound stain; and
(e) detecting and quantifying light emission from the bound stain;
wherein the bacteria-binding apparatus surface specifically binds mycobacteria or a phylogenetic group of mycobacteria, or the stain specifically stains mycobacteria or a phylogenetic group of mycobacteria; and wherein light emission above a threshold value is diagnostic of mycobacterial infection.

2. The method of claim 1 wherein the isolated mycobacteria are contacted with the stain solution and washed with the wash solution while bound to the bacteria-binding apparatus surface, and the light emission is detected and quantified from the bacteria-binding apparatus surface.

3. (canceled)

4. The method of claim 1 wherein steps (b) through (e) are performed in less than 10 minutes.

5. The method of claim 1 wherein the mycobacteria from the sputum sample are not amplified by culturing.

6-7. (canceled)

8. The method of claim 1 wherein the surface that bindings mycobacteria or a phologenetic group of mycobacteria is a surface of bacteria-binding beads, and wherein the fluorescent or chemiluminescent stain solution and the wash solution are stacked zones in a cartridge, wherein the cartridge includes a pump mechanism and a receptacle for receiving the beads, and the stacked zone solutions are pumped sequentially across the beads by the pump mechanism in the cartridge.

9-11. (canceled)

12. The method of claim 1 wherein the fluorescent or chemiluminescent stain is an acid-fast fluorescent or chemiluminescent stain that specifically stains mycobacteria.

13. (canceled)

14. The method of claim 1 wherein the fluorescent or chemiluminescent stain is a fluorescent or chemiluminescent group conjugated to an antibody that specifically recognizes mycobacteria or a phylogenetic groups of mycobacteria.

15-20. (canceled)

21. A handheld disposable cartridge for use in detecting mycobacteria comprising:
a receptacle holding a bacteria-binding apparatus having a surface that binds mycobacteria; and
a fluorescent or chemiluminescent stain that stains mycobacteria functionally coupled to the receptacle to stain mycobacteria held by the apparatus in the receptacle;
wherein the bacteria-binding apparatus surface specifically binds mycobacteria or a phylogenetic group of mycobacteria, or the stain specifically stains mycobacteria or a phylogenetic group of mycobacteria.

22. The cartridge of claim 21 wherein the stain specifically stains mycobacteria or M. tuberculosis.

23. The cartridge of claim 22 wherein the fluorescent or chemiluminescent stain is an acid-fast stain.

24. The cartridge of claim 23 wherein the acid-fast stain is auramine O-rhodamine.

25. The cartridge of claim 22 wherein the stain comprises a fluorescent or chemiluminescent dye coupled to an antibody that specifically binds mycobacteria or M. tuberculosis.

26. The cartridge of claim 21 wherein the bacteria-binding apparatus includes beads having the surface that binds mycobacteria.

27-31. (canceled)

32. A kit for detecting mycobacteria comprising packaging material containing:
(a) a disposable sputum receptacle comprising:
(i) a vessel for receiving sputum; and
(ii) a bacteria-binding apparatus functionally coupled to the vessel and having a surface that binds mycobacteria;
(b) a handheld disposable cartridge for use in detecting mycobacteria comprising:
(i) a receptacle for holding the bacteria-binding apparatus having a surface that binds mycobacteria; and
(ii) a fluorescent or chemiluminescent stain that stains mycobacteria functionally coupled to the receptacle to stain mycobacteria held by the apparatus in the receptacle and
wherein the fluorescent or chemiluminescent stain specifically stains mycobacteria or a phylogenetic group of mycobacteria or the bacteria-binding apparatus surface specifically binds mycobacteria or a phylogenetic group of mycobacteria.

33. The kit of claim 32 wherein the bacteria-binding apparatus comprises a collection of beads having a surface that binds mycobacteria or a phylogenetic group of mycobacteria.

34-35. (canceled)

36. The kit of claim 32 wherein the stain is auramine O-rhodamine B.

37. A method of determining whether a person afflicted with mycobacteria is infected with drug-resistant or drug-sensitive mycobacteria comprising:
(a) obtaining a sputum sample from the person;
(b) dividing the sputum sample or mycobacteria isolated from the sputum sample into 2 or more mycobacteria-containing fractions containing approximately equal numbers of mycobacteria;
(c) placing one of the mycobacteria-containing fractions into a control vessel containing a selective mycobacterial growth medium that allows growth of mycobacteria and inhibits growth of non-mycobacteria, wherein the medium lacks any test agent;
(d) placing each of the one or more other mycobacteria-containing fractions into a test vessel containing the selective medium supplemented with one or more test anti-mycobacteria agents;
(e) incubating the control and test vessels for a period of time sufficient to allow mycobacterial growth in at least the control vessel; and
(f) assaying for mycobacterial cell numbers in the control and test vessels by a process comprising:
(i) lysing mycobacteria from the vessels to release mycobacterial ATP, and contacting mycobacterial ATP with luciferin and luciferase in an assay solution for each vessel; and
(ii) detecting and quantifying light emitted from the assay solution for each vessel, wherein the quantity of light emitted is proportional to the number of mycobacterial cells in the vessel after the growth period.

38. The method of claim 37 wherein step (b) comprises contacting the sputum sample with a plurality of particles having a surface that binds mycobacteria, and dividing the plurality of particles into two or more groups of approximately equal total surface area of particles, wherein the two or more groups of particles contain approximately equal numbers of bound mycobacteria, and wherein the particles are the mycobacteria-containing fractions placed into the vessels containing growth medium in steps (c) and (d).

39. The method of claim 37 wherein the cells are lysed by contacting the mycobacterial cells with a solution comprising a lysing agent, the luciferin, and the luciferase.

40. The method of claim 37 wherein in step (e) the vessels are incubated for 3 days or less.

41. The method of claim 37 wherein one of the mycobacteria-containing fractions is placed into a test vessel containing selective medium supplemented with rifampin and another of the mycobacteria-containing fractions is placed into a test vessel containing selective medium supplemented with isoniazid, or one of the mycobacteria-containing fractions is placed into a test vessel containing selective medium supplemented with isoniazid and rifampin.

42-55. (canceled)
